(19) United States
(12) Patent Application Publication
(10) Pub. No.: US 2018/0153660 A1
(43) Pub. Date: Jun. 7, 2018

(54) PROCESS FOR THE PREPARATION OF A TOPOGRAPHY FOR IMPROVED PROTEIN ADHERENCE ON A BODY MADE OF TITANIUM OR A TITANIUM ALLOY

(71) Applicant: STRAUMANN HOLDING AG, Basel (CH)

(72) Inventor: Simon BERNER, Basel (CH)

(73) Assignee: STRAUMANN HOLDING AG, Basel (CH)

(21) Appl. No.: 15/576,427

(22) PCT Filed: May 26, 2016
(86) PCT No.: PCT/EP2016/061934

§ 371 Date: Nov. 22, 2017

(30) Foreign Application Priority Data
May 26, 2015 (GB) 1508955.0

(51) Int. Cl. A61C 13/00; C23C 22/64; A61C 8/00; A61K 6/00; A61K 6/04; A61L 27/06; A61L 27/50; C23C 22/68; C23F 1/38

(52) U.S. Cl. CPC A61C 13/0006; C23C 22/64; A61C 8/0015; A61K 6/0017; A61K 6/04; A61C 2008/0046; A61L 27/50; C23C 22/68; C23F 1/38; A61L 2400/18; A61L 27/06

(57) ABSTRACT

A process for the preparation of a topography for improved protein adherence on a body made of titanium or a titanium alloy wherein the process includes the subsequent steps of pre-treating the surface including etching the surface with an etching solution containing a mineral acid, and forming on the pre-treated surface obtained under titanate-including sub-microscopic structures by treating the pre-treated surface with an aqueous solution containing an oxidative agent, the sub-microscopic structures extending in at least two dimensions to 1 μm at most.

The present invention relates to a process for the preparation of a topography for improved protein adherence on a body made of titanium or a titanium alloy, to a body obtainable by this process as well as to the use of the body for a dental implant or a dental implant abutment.

Implants, such as dental implants, are well known in the art. They generally consist of a material, which is biocompatible and which additionally has favourable mechanical properties. Currently used dental implants are often made of titanium or a titanium alloy, which apart from being biocompatible exhibit outstanding mechanical strength.

The acceptance of the human body towards an implant is determined by the implant surface. When detected as a foreign object and rejected by the immune system, the implant may cause inflammation, which not only causes pain to the patient but often also leads to the necessity of a second surgery to remove or replace the implant.

In order to avoid rejection of the implant by the human body, the implant surface must be engineered in a manner that cells attach to it and that natural body tissue, specifically bone tissue or soft tissue, start growing around the implant.

In the case of a dental implant, for example, it is required that a direct structural and functional connection between living jaw bone and the implant surface is achieved shortly after implantation. This is referred to in the art as “osteointegration” (or “osteointegration”): a good osteointegration means that the implant safely ossifies within a short healing time so that a permanent bond between implant and bone is obtained.

Besides the importance of the osteointegrative properties, there is increasing evidence that also a good interaction between the dental implant and the surrounding supracrestal connective tissue (in the following referred to as the “soft tissue”) is crucial for a successful implantation. This is supported by the view that the soft tissue plays a fundamental role in establishing an effective seal between the oral environment and the endosseous part of a dental implant and, thus, also a barrier for bacteria to adhere on the soft tissue contact surface and the bone tissue contact surface of the implant.

The attachment of cells of the surrounding soft or bone tissue is governed by proteins which adhere, i.e. adsorb, to the surface once the implant gets in contact with blood. It is assumed that the proteins adsorbed on the implant surface influence the behaviour, e.g. the differentiation, of the cells of the respective tissue.

In order to achieve a fast and strong interaction between the dental implant and the respective tissue, adherence of these proteins on the surface is thus of paramount importance.

One important factor that influences protein adherence is the hydrophilicity of the surface.

Recently, it has been found that also the presence of specific nanostructures may play an important role in the adherence of proteins.

Specifically, WO2013/056844 describes a process for providing structures for an improved protein adherence on the surface of a body, specifically an implant. The process comprises the step of storing an acid-etched basic body in an aqueous solution, by which nanostructures are formed on the surface of the basic body.

Further, R. A. Gittens et al. (Biomaterials 32 (2011) 3395-3403) report on studies focussing on the hierarchical combination of both micro- and nanoscale roughness to promote osseointegration on clinically-relevant surfaces.

Notwithstanding the good results achieved according to the process of WO2013/056844, there is an ongoing need for providing a body having a topography of improved protein adherence, ultimately allowing for a fast and strong interaction of the body with the surrounding tissue.

In consideration of this, the object to be solved by the present invention is to provide a process for preparing a topography on a body made of titanium or a titanium alloy in a manner to allow for an improved adherence of proteins. In particular, a simple and reproducible process for modifying the body's surface shall be provided which allows for an improved adherence of at least one blood protein mediating blood coagulation and/or cell attachment and, thus, tissue interaction with the body.

The object of the present invention is solved by the process according to claim 1. Preferred embodiments of the invention are defined in the dependent claims.

According to claim 1, the process of the present invention is directed to the preparation of a topography for improved protein adherence on a body made of titanium or a titanium alloy, i.e. the most common material used for dental implants or dental implant abutments.

The process comprises the subsequent steps of
a) etching the surface with an etching solution comprising a mineral acid, and
b) forming on the etched surface obtained under a) titanate-comprising sub-microscopic structures by treating the etched surface with an aqueous solution comprising an oxidative agent, the sub-microscopic structures extending in at least two dimensions to 1 μm at most.

It has surprisingly been found that by the process of the present invention, in particular the titanate-comprising sub-microscopic structures formed in step b), a particularly pronounced and specific adherence of proteins mediating blood coagulation and/or cell attachment can be achieved. Ultimately, a fast blood coagulation and, hence, formation of the fibrin network and/or a strong attachment of the cells of the surrounding tissue can, thus, be achieved, which in particular can lead to an improved osteointegration of the body.

Without wanting to be bound by the theory, the effect of improved cell attachment can be explained by the following mechanism:

When a body is implanted into tissue, particularly into bone tissue, it is first contacted by water molecules from the surrounding blood. In a next step, ions and proteins will accumulate and adhere on the implant's surface, but without actually penetrating the material. As mentioned above, this “protein adherence” or “protein adsorption” is assumed to be decisive for later cell responses.

By the specific topography obtainable by the present invention, in particular the titanate-comprising sub-microscopic structure, “protein retention structures” are provided on the body's surface, i.e. structures which allow an improved adherence of specific proteins.

This will be further shown by the attached working examples below, according to which the topography allows for a relatively selective adherence of fibrinogen and fibronectin, i.e. proteins to which an important role in the blood coagulation and, hence, the formation of the fibrin network is attributed.

Given the formation of sub-microscopic structures in step b), the process of the present invention is in any respect different from the technology described in “Alkali Treatment of Microrough Titanium Surfaces Affects Macrophage/Monocyte Adhesion, Platelet Activation and Architecture of Blood Clot Formation” by V. Milleret et al., European Cells and Materials Vol. 21 2011, pp 430-444, describing the treatment of a titanium surface in a manner that the surface topography is not affected.

The process of the present invention is also in clear contrast to any process devoid of step a), i.e. any process in which no etching is performed, as it is the case for the process described in WO 2009/024778. Notwithstanding the mechanism described above, according to which proteins accumulating and adhering on the surface put the surface in an improved condition for later cell response, topographical features as received by etching the surface in step a) of the present invention have nevertheless been found to form an essential basis for achieving good osteointegration.

The term “sub-microscopic structures” as used in the context of the present invention is to be interpreted broadly and relates to any structure, typically a particulate structure, more particularly a crystalline structure, which extends in at least two dimensions to 1 μm at most. The term is equivalent to the term “sub-micron structures”. Although the structure is typically crystalline, as mentioned, this has not necessarily to be the case. Hence, structures, which are partially crystalline, but not solely crystalline, are also encompassed by the term “sub-microscopic structures”.

Given the particulate nature of the sub-microscopic structure according to the present invention, it is in contrast to any structure obtained by nanopitting, as e.g. described in US 2011/233169.

Typically, a layer comprising or essentially consisting of the sub-microscopic structures is formed in step b), the layer forming a sub-microscopic topographical formation formed on a microscopic topographical formation formed in step a). Thus, a formation of different topographical scale is formed in step b) than in step a), leading to a hierarchical topography of the body.

Specifically, the layer has a thickness of more than 10 nm, which is apparent from the fact that no metallic titanium signal is detected by X-ray photoelectron spectroscopy (XPS). The layer is thus thicker than an oxide layer spontaneously formed on titanium surfaces in the presence of air.

Unlike step a), which is a subtractive process step, step b) is an additive process step. The process of the present invention, thus, includes the removal of material from the body in step a), combined with the addition of material in step b).

As will be further discussed in detail below, subtractive process step a) preferably corresponds to the acid etching according to the well-known SLA® treatment. Specifically, step a), thus, relates to a pre-treatment comprising a mechanical subtractive treatment, more particularly a sand-blasting treatment, prior to the etching.

According to a preferred embodiment of the present invention, the microscopic topographical formation formed in step a) is defined by at least one of the following surface parameters:
i) Sₐ being the arithmetic mean deviation of the surface in three dimensions and being in the range from 0.1 μm to 2.0 μm, preferably being in a range from 0.4 μm to 1.8 μm, more preferably from 0.8 μm to 1.7 μm, and most preferably from 0.9 μm to 1.5 μm;
ii) Sₜ being the maximum peak to valley height of the profile in three dimensions and being in the range from 1.0 μm to 20.0 μm, preferably being in a range from 3.0 μm to 18.0 μm, more preferably from 4.5 μm to 13.0 μm, and most preferably from 6.0 μm to 12.0 μm; and/or
iii) Sₛₖ being the skewness of the profile in three dimensions and being in the range from −0.6 to 0.6, preferably from −0.4 to 0.6, more preferably from −0.3 to 0.5.

The surface parameters are known to the skilled person and are analogue parameters for three dimensions to the parameters Rₐ, Rₜ and Rₛₖ, respectively, defined in EN ISO 4287 for two dimensions. Specifically, the above values relate to the values as e.g. obtainable by the WinSAM software (SAM (Surface Analysis Method) for Windows) known to the skilled person.

The above values for Sₐ, Sₜ and Sₛₖ relate in particular to a bone contacting surface of the body, i.e. a surface area located such on the body, specifically the implant, to come into contact with bone tissue after implantation. For a soft tissue contacting surface of the body, the preferred values are smaller. Specifically, Sₐ is preferably in the range from 0.05 μm to 0.5 μm, more preferably from 0.05 μm to 0.3 μm for a soft tissue contacting surface.

As mentioned, the microscopic topographical formation defined above is typically obtained by step a) further comprising a sand-blasting treatment prior to the etching.

Regarding the microscopic topographical formation, the surface parameters are preferably in the range of “SLA®” or “SLActive®” surfaces, given the above mentioned preferred embodiment in which the etching according to a) is performed according to the SLA® or SLActive® protocol.

Both the “SLA®” and “SLActive®” treatment are well-known in the respective field and relate to a breakthrough technology in view of the preparation of osteophilic implants. Specifically, “SLA®” involves sandblasting the implant's surface followed by treating it with an etching solution comprising a first mineral acid, whereas “SLActive®” further comprises conditioning the “SLA” surface either in nitrogen or in an isotonic saline solution, thereby maintaining the high hydrophilicity of the “SLA®” surface which would otherwise be lost during storage due to interaction with the atmosphere.

According to a preferred embodiment of the present invention, the first etching solution, thus, comprises or essentially consists of a mixture of HCl and H₂SO₄. More particularly, a mixture of HCl and H₂SO₄ at a temperature higher than 80° C. is used for step a). Alternatively, any other solution of at least one mineral acid can be used for process step a), in particular a solution comprising at least one mineral acid selected from the group consisting of HCl, H₂SO₄, H₃PO₄ and mixtures thereof.

It is further preferred that prior to step a) a macroscopic topographical formation is provided to the surface, more preferably by sand-blasting. For example, corundum having a particle size from 250-500 μm can be used as blasting material. In this embodiment, also the sand-blasting step of the SLA® technology is applied to the process of the present invention.

According to a specifically preferred embodiment, the present invention, thus, involves the same steps as according to the SLA® protocol, but further comprising step b) after the SLA® etching step.

Preferably, the process is carried out in a manner that by the sub-microscopic topographical formation formed in step b), at least one surface parameter defining the microscopic topographical formation formed in step a) and being selected from the group consisting of Sₐ, Sₜ and Sₛₖ is changed by 50% at most, preferably by 30% at most, more preferably by 20% at most and most preferably is kept essentially unchanged.

Thus, the well-established macroscopic and microscopic topographical formation according to the SLA® technology is not altered or only altered to a negligible degree by process step b). This will be further illustrated by the attached figures showing an almost identical picture at a magnification focussing on the macroscopic and microscopic topographical formation of the surface, but showing a completely different picture of the surface at a magnification focussing on the sub-microscopic topographical formation.

Depending on the specific material of the body and on the process parameters, different sub-microscopic structures can be obtained. Specifically, the sub-microscopic structures extend to a length of more than 50 nm, preferably more than 100 nm, more preferably more than 150 nm.

As mentioned previously, the sub-microscopic structures are typically crystalline.

According to a specific embodiment, at least some of the sub-microscopic structures have the shape of a lamella, sheet and/or wire, as will be illustrated by way of the attached figures discussed below. In this regard, it is particularly preferred that the thickness of the lamella, sheet and/or wire is in the range from 1 to 50 nm, preferably 5 to 30 nm, more preferably 10 to 20 nm, and the lamella and/or wire extend in direction perpendicular to the direction of thickness from 50 to 1000 nm, preferably 50 to 600 nm, more preferably 75 to 500 nm and most preferably 100 to 500 nm.

More specifically, the lamellae and/or wires are entangled with each other, thereby forming a network of structures. This is of particular interest in view of protein adsorption, since by the network, cellular structures are formed with the lamellae and/or wires forming the walls or scaffolds enclosing the interior of these cellular structures. Without wanting to be bound by the theory, the formation of cellular structures can further contribute to the retention or “entrapment” of proteins, ultimately allowing for a strong and specific adsorption of the proteins of interest.

It has surprisingly been found that the sub-microscopic structures are stable in storage media and, in particular, are maintained after storing the body in NaCl solution or in air.

It has also been found, that on a surface which is hydrophilic (or superhydrophilic) the sub-microscopic structures formed according to the present invention effect maintenance of the hydrophilicity or superhydrophilicity even after prolonged storage in air, which would otherwise lead to the surface becoming hydrophobic.

Depending on the specific shape to be achieved, the process parameters are to be chosen respectively.

According to a specifically preferred embodiment, the treatment under step b) is performed under elevated temperatures, preferably a temperature above 30° C., more preferably above 40° C. and most preferably above 100° C.

It is preferred that the oxidative agent is an inorganic hydroxide. More preferably, it is a hydroxide of an alkali metal, in particular sodium hydroxide and/or potassium hydroxide, and/or a hydroxide of an alkaline earth metal, in particular calcium hydroxide, magnesium hydroxide and/or strontium hydroxide. Sodium hydroxide and/or potassium hydroxide are particularly preferred. Alternatively or additionally, hydrogen peroxide can be used as oxidative agent.

It is further preferred that the concentration of the oxidative agent is in the range from 0.1 to 5 M, preferably from 0.3 to 3 M, most preferably from 0.5 to 2 M. The concentration is thus much higher than what is taught in the above mentioned article of Milleret et al., according to which 0.05 M aqueous NaOH was used.

According to a further preferred embodiment, the treatment in step b) is carried out for a duration in the range from 0.25 hours to 12 hours, preferably from 0.25 hours to 5 hours, more preferably from 0.5 hours to 2.5 hours. The duration is thus much longer than what is taught in the above mentioned article of Milleret et al., according to which the treatment was carried out for 30 seconds only.

By choosing the process parameters according to at least one of these preferred embodiments, titanate-comprising sub-microscopic structures exhibiting a particularly high capability for protein adsorption, in particular fibrinogen and/or fibronectin adsorption can be obtained. In this regard it is particularly preferred that in step b) the etched surface obtained in step a) is treated with an aqueous solution of sodium hydroxide and/or potassium hydroxide such that sodium titanate-comprising sub-microscopic structures or potassium titanate-comprising sub-microscopic structures are formed, respectively. For example, treatment in an aqueous solution comprising 1 M NaOH at 124° C. for 2 hours leads to the preferred lamellar and/or wire-like structures that are entangled with each other.

After the treatment according to step b), the body is preferably stored in an aqueous solution comprising the oxidative agent, but at a lower concentration than used for the treatment according to step b). For the above mentioned specific example using an aqueous solution comprising 1 M NaOH, the body can e.g. be stored in 0.01 M NaOH. Alternatively, the body can also be stored in e.g. 0.9% NaCl solution or in air.

According to a further preferred embodiment, the body is made of a titanium-zirconium alloy, since for this material a surface topography of particular relevance can be achieved by the process of the present invention. More preferably, the body is made of a bimetallic titanium-zirconium alloy comprising 13-17% zirconium. A particularly preferred titanium-zirconium alloy is available under the tradename Roxolid® (Institut Straumann AG, Switzerland), the properties of which being well-known to the person skilled in the art. Alternatively, the body is made of titanium, for which also a surface topography of high relevance can be achieved by the process of the present invention. Depending on the aim to be achieved, the body can also be made of titanium, since it has been found that also for a body made of titanium, in particular a titanium implant, a topography for improved protein adherence can be achieved by the process of the present invention. Specifically, the morphology of the sub-microscopic structures obtained can be governed by the choice of the body's material.

As pointed out above, the process of the present invention is in particular directed to a dental implant or a dental implant abutment, in order to provide it with a surface that allows for a strong interaction with the surrounding tissue. According to a preferred embodiment, the body is a dental implant or a dental implant abutment and the topography is provided on at least a portion of the surface of the body that in use is intended to be in contact with bone tissue or soft tissue, respectively.

Apart from the process described above, the present invention also relates to a body obtainable by the process.

Specifically, the invention relates to a body, the surface of which being defined by a microscopic topographical formation and a sub-microscopic topographical formation formed on the microscopic topographical formation, said sub-microscopic topographical formation comprising or essentially consisting of a layer of titanate-comprising sub-microscopic structures extending in at least two dimensions to 1 μm at most.

As mentioned above, the microscopic topographical formation is defined by at least one of the following surface parameters:
i) Sₐ being the arithmetic mean deviation of the surface in three dimensions and being in the range from 0.1 μm to 2.0 μm, preferably being in a range from 0.4 μm to 1.8 μm, more preferably from 0.8 μm to 1.7 μm, and most preferably from 0.9 μm to 1.5 μm;
ii) Sₜ being the maximum peak to valley height of the profile in three dimensions and being in the range from 1.0 μm to 20.0 μm, preferably being in a range from 3.0 μm to 18.0 μm, more preferably from 4.5 μm to 13.0 μm, and most preferably from 6.0 μm to 12.0 μm; and/or
iii) Sₛₖ being the skewness of the profile in three dimensions and being in the range from −0.6 to 0.6, preferably from −0.4 to 0.6, more preferably from −0.3 to 0.5.

Specifically, the above values relate to the values as e.g. obtainable by the WinSAM software (SAM (Surface Analysis Method) for Windows) known to the skilled person, as mentioned above.

It is understood that all features which are described above as preferred features of the process likewise are preferred features of the body of the present invention and vice versa.

According to a still further aspect, the present invention also relates to the use of the body according to any of the preceding claims for a dental implant or a dental implant abutment. In this regard, the body can be used as the dental implant or the dental implant abutment or as a part of the dental implant or dental implant abutment. It is understood that when the body is used as a part of the dental implant or dental implant abutment, at least a portion of the remaining part can be made of a material other than titanium or titanium alloy, respectively.

If the body is used as a dental implant abutment, the values for Sₐ, Sₜ and Sₛₖ are preferably lower than the ones mentioned above, which in particular relate to a bone contacting surface of the body. Specifically, Sₐ is in the case of the body being a dental implant abutment preferably in the range from 0.05 μm to 0.5 μm, and more preferably from 0.05 μm to 0.3 μm. This allows a particularly strong interaction of the dental implant abutment and the surrounding soft tissue to be obtained.

### EXAMPLES

### 1. Examples Relating to In Vitro-Analysis

1.1. Materials and Methods

Material

Discs, 5 mm in diameter and 1 mm in thickness, were prepared from a bimetallic TiZr alloy rod (Roxolid (RXD); 13-17% Zr).

“SLA” Treatment

First, the samples have been treated according to the protocol for preparing “SLA®” samples. Specifically, the samples have been sand-blasted using corundum with large grits (particle size 250-500 μm), followed by etching the sand-blasted surface in a boiling mixture of HCl and H₂SO₄.

Samples “RXD SLActive”

The samples achieved by the SLA® treatment have been directly immersed and stored in 0.9% NaCl solution, according to the SLActive® protocol.

For comparative reasons, a first portion of samples have been kept without further treatment. The samples are hereinafter referred to as “RXD SLActive”.

Samples “RXD SLActive NaOH” and “RXD SLActive NaOH—NaCl”

A second portion of the samples achieved according to the SLActive® protocol have then been transferred into a Teflon beaker and treated in an aqueous solution comprising 1 M NaOH at 124° C. for 2 hours. Thereby, titanate-comprising sub-microscopic structures being in the form of lamellae entangled with each other are formed.

The samples have then been rinsed and neutralized by placing them in a large beaker containing 400 ml ultrapure water. A first set of samples was then stored in 0.01 M NaOH and γ-sterilized, whereby samples “RXD SLActive NaOH” according to the present invention have been achieved. Instead of storing in NaOH, a second set of samples has been stored in 0.9% NaCl to produce samples “RXD SLActive NaOH—NaCl”.

Samples “RXD SLA NaOH (Storage in Air)”

Freshly prepared “RXD SLActive” samples have been treated in an aqueous solution comprising 1 M NaOH at 124° C. for 2 hours, then rinsed and neutralized, as described above.

The samples were then packed dry in air, γ(gamma)-sterilized and then stored in air for 8 months, whereby samples “RXD SLA NaOH (storage in air)” have been achieved.

1.2. Evaluation Methods

1.2.1. Contact Angle Measurements

Contact angle measurements were performed in order to determine the degree of hydrophilicity or hydrophobicity. For “RXD SLActive NaOH” and “RXD SLActive”, three sample discs were analysed.

The contact angles were determined using a sessile drop test with ultrapure water (EasyDrop DSA20E, Krüss GmbH). A droplet size of 0.1 μl (microliter) was used and the samples were blown dry in a stream of Ar prior to the contact angle measurements. Contact angles were calculated by fitting a circular segment function to the contour of the droplet on the surface.

The results of the contact angle measurements are given in Table 1.

TABLE 1
Contact angles

| | disc 1 | disc 2 | disc 3 |
|---|---|---|---|
| | CA [°] | CA [°] | CA [°] |
| RXD SLActive NaOH | 0 | 0 | 0 |
| RXD SLActive | 0 | 0 | 0 |
| RXD SLA NaOH (storage in | 0 | 0 | n.n. |
| air) | | | |

As shown in Table 1, both the RXD SLActive NaOH and the RXD SLActive samples were superhydrophilic exhibiting contact angles of 0°.

Further experiments have shown that superhydrophilicity was maintained even after storing the RXD SLActive NaOH sample for 1 month in air.

Surprisingly, even the RXD SLA NaOH (storage in air) samples have been found to be superhydrophilic. Thus, the sub-microscopic structures formed according to the present invention effect maintenance of the superhydrophilicity of the samples even after prolonged storage in air, which would otherwise lead to the samples becoming hydrophobic.

Also, no substantial change in the topography could be determined after this storage period in air, as will be shown below.

1.2.2. SEM (Scanning Electron Microscopy) and EDX (Energy Dispersive X-Ray Spectroscopy)

The visual appearance and morphology of the nanostructures were evaluated with scanning electron microscopy (SEM).

SEM measurements were performed on three discs for each type of surface. The measurements were performed on a scanning electron microscope of the type Zeiss Supra 55. The overview SEM images were acquired with an acceleration voltage of 20 kV using the Everhart-Thornley detector and the high-resolution images with an acceleration voltage of 5 kV using the in-lens detector.

Further, EDX spectra were acquired in case of the RXD SLActive NaOH sample in order to analyse the chemical composition and constitution of the sample surface.

The SEM images of the samples and the EDX spectra of the RXD SLActive NaOH sample are given in the attached figures, whereby

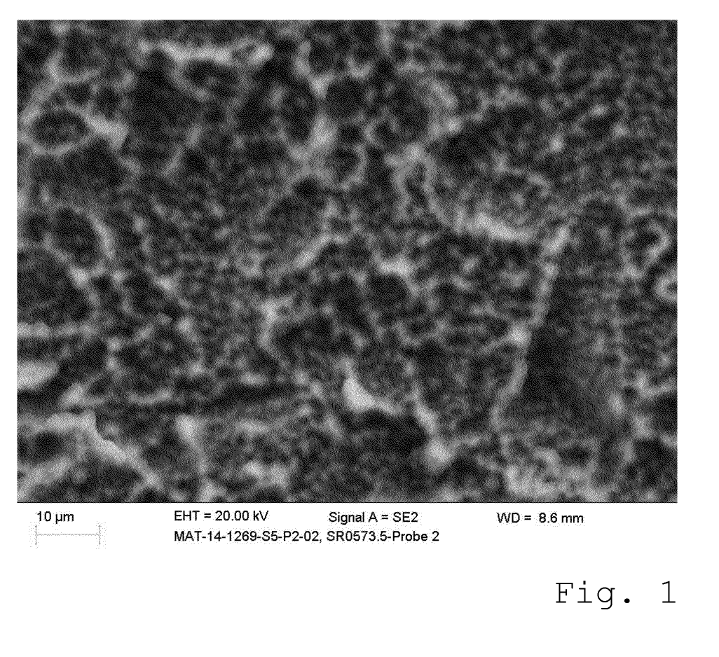

FIG. 1 relates to a SEM image of the surface of sample RXD SLActive NaOH in a magnification of about 1′000×, the scale corresponding to 10 micrometer being given in the bottom left corner of the image;

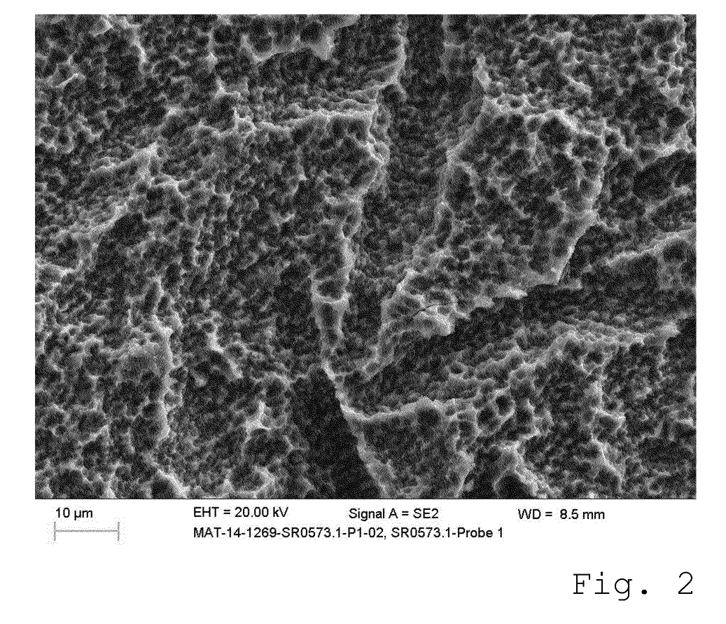

FIG. 2 relates to a SEM image of the surface of sample RXD SLActive in a magnification of about 1′000×, the scale corresponding to 10 micrometer being given in the bottom left corner of the image;

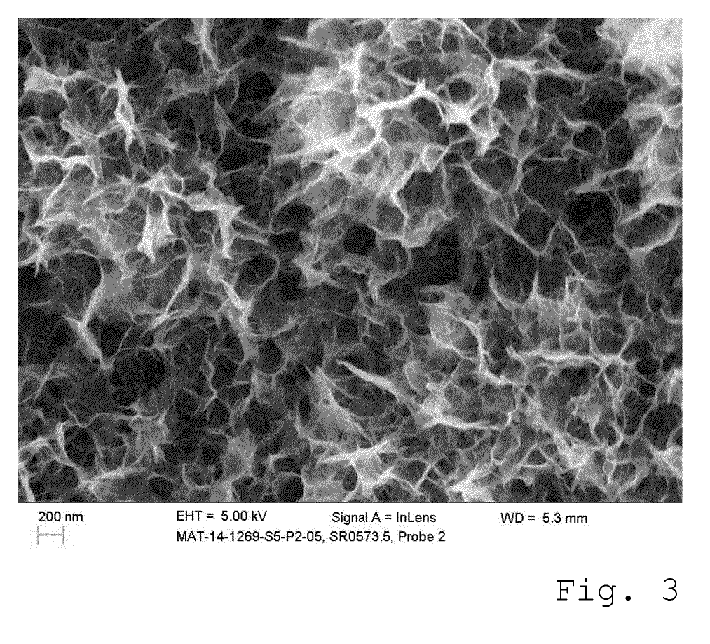

FIG. 3 relates to a SEM image of the surface of sample RXD SLActive NaOH in a magnification of about 20′000×, the scale corresponding to 200 nanometer being given in the bottom left corner of the image;

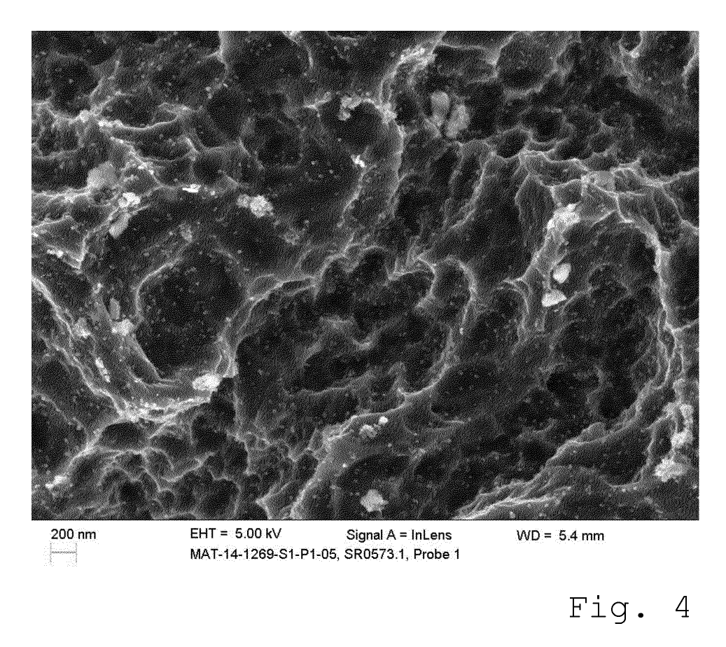

FIG. 4 relates to a SEM image of the surface of sample RXD SLActive in a magnification of about 20′000×, the scale corresponding to 200 nanometer being given in the bottom left corner of the image;

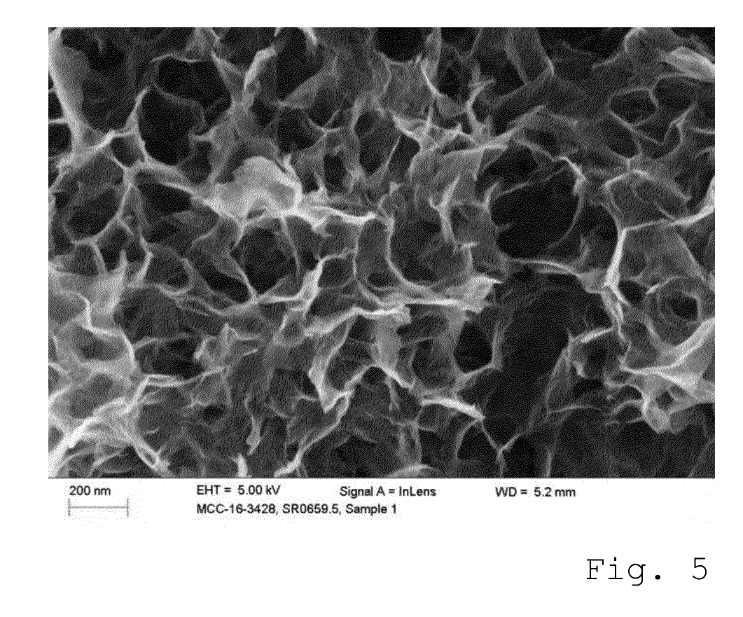

FIG. 5 relates to a SEM image of the surface of sample RXD SLA NaOH (storage in NaCl) in a magnification of about 50′000×, the scale corresponding to 200 nanometer being given in the bottom left corner of the image;

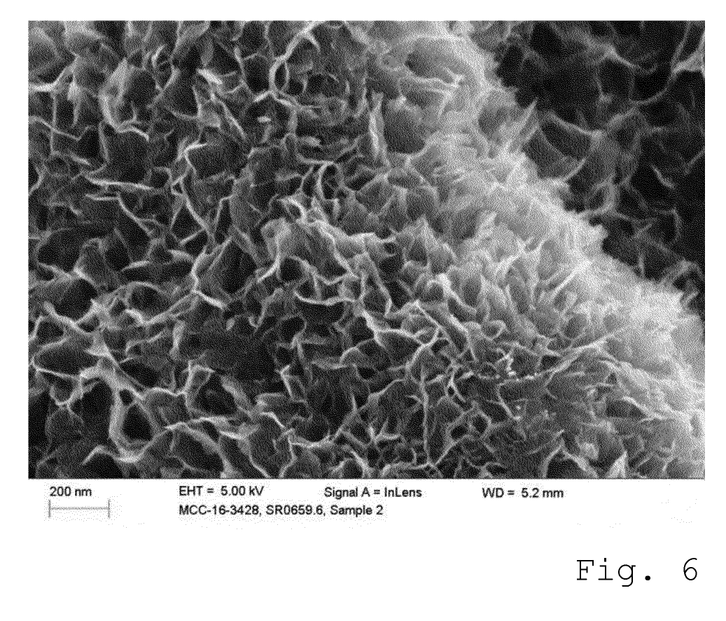

FIG. 6 relates to a SEM image of the surface of sample RXD SLA NaOH (storage in air) in a magnification of about 50′000×, the scale corresponding to 200 nanometer being given in the bottom left corner of the image; and

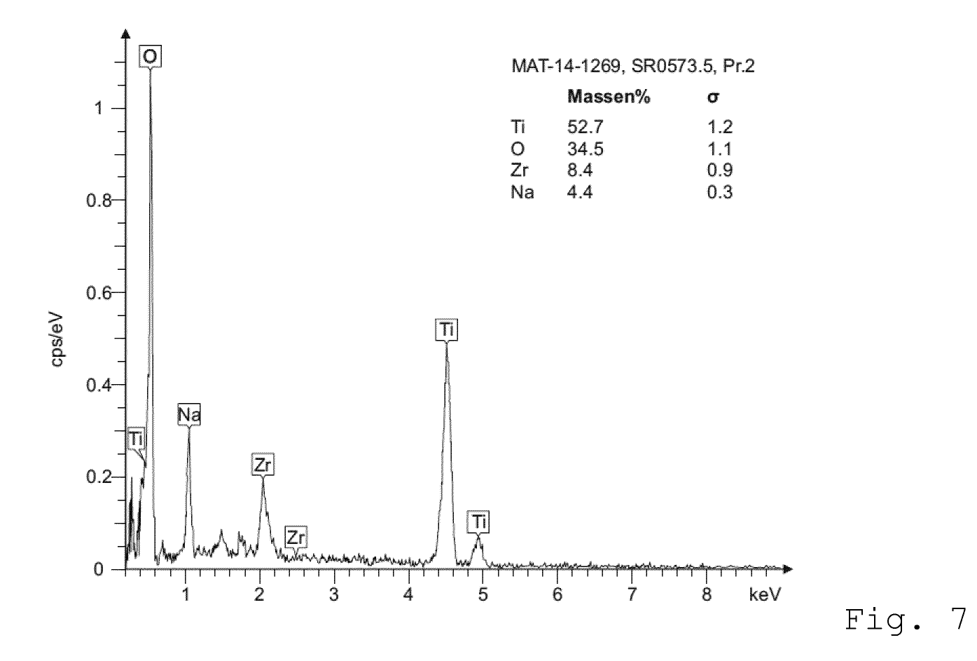

FIG. 7 relates to the EDX spectrum obtained on a 200× image of R×D SLActive NaOH.

As can be seen from FIGS. 1 to 4, the RXD SLActive and RXD SLActive NaOH samples exhibit the macroscopic and microscopic topographical formation obtained by the SLA® treatment, namely by sandblasting and etching the samples in a boiling mixture of HCl and H₂SO₄.

However, there are distinct differences in the appearance of a sub-microscopic topographical formation between sample RXD SLActive and RXD SLActive NaOH. Specifically, sub-microscopic structures in the form of lamellae or wires entangled with each other and, thus, forming a network are visible for sample RXD SLActive NaOH at the respective magnification shown in FIG. 3, whereas such sub-microscopic structures are not visible for sample RXD SLActive shown in FIG. 4. Further experiments have shown that the sub-microscopic topographical formation is maintained after storing the RXD SLActive NaOH samples in NaCl (FIG. 5) or in air (FIG. 6). In particular, FIG. 6 shows that the RXD SLA NaOH sample, which has been stored for 8 months in air, exhibits a very similar sub-microscopic topographical formation as the one of the RXD SLActive NaOH sample shown in FIG. 3.

As further shown by the EDX spectrum according to FIG. 7, the RXD SLActive NaOH sample has a relatively thick oxide layer thickness indicated by the pronounced signal for oxygen. Also, the RXD SLActive NaOH sample shows a pronounced signal for sodium, both signals being indicative of sodium-titanate comprising sub-microscopic structures. As a result of i.a. the increased oxide-layer thickness, the SEM image of the RXD SLActive NaOH sample shown in FIG. 1 is blurred.

Further experiments have shown that the oxide layer thickness is in the range from 500 nm to 800 nm and that sodium is observed to a depth of about 800 nm.

1.2.3. Roughness Parameter Determination

Roughness images were acquired using a confocal microscope (psurf explorer, NanoFocus AG, Oberhausen, Germany) equipped with a 20× lens. Three measurements were performed on each sample disc and three discs were measured for each type of surface. The roughness parameters were calculated using the WinSAM software mentioned above. The whole roughness image with a size of 798 μm (micrometer)×798 μm (micrometer) was used for the calculation of the 3D roughness parameters.

The values of the microscopic topographical formation (roughness) were determined using a moving average Gaussian filter with a cut-off wavelength of 30 μm (x=31 μm, y=30 μm, 20×19 image points). Then, the roughness parameters were calculated by means of a KFL analysis with limits from the amplitude density.

Specifically, Sₐ (the arithmetic mean deviation of the surface in three dimension), Sₜ (the maximum peak to valley height of the profile in three dimensions) and Sₛₖ (the skewness) were determined in analogy to EN ISO 4287 relating to the respective parameters Rₐ, Rₜ and Rₛₖ in two dimensions. For the parameters in three dimensions, it is further referred to ISO 25178, in which the symbol S_{z} is used for the maximum peak to valley height of the profile (instead of the symbol Sₜ used in the context of the present invention).

Table 2 presents the mean values of the microroughness values of the two samples. The table shows that at least the values of Sₐ and Sₜ of both RXD SLActive and RXD SLActive NaOH lie within the same range typically observed for SLA®/SLActive® implants. Similar values are obtained for RXD SLActive NaOH samples after storing in 0.9% NaCl solution or after storing in air for 1 month. Specifically, the Sₐ, Sₜ and Sₛₖ values of R×D SLActive NaOH deviate from the respective values of the RXD SLActive samples by less than 25%.

TABLE 2
Values of the microscopic topographical formation

| | Sa | Std Sa | St | Std St | | Std |
|---|---|---|---|---|---|---|
| | [μm] | [μm] | [μm] | [μm] | Ssk | Ssk |
| RXD | 1.183 | 0.037 | 7.91 | 0.31 | 0.215 | 0.041 |
| SLActive | | | | | | |
| NaOH | | | | | | |
| RXD | 0.970 | 0.046 | 6.38 | 0.26 | 0.192 | 0.051 |
| SLActive | | | | | | |

1.2.4. Protein Adsorption Measurements

Albumin (from bovine serum (BSA), Alexa Fluor 647 conjugate, Invitrogen, USA), fibrinogen (from human plasma; HPF, Alexa Fluor 546 conjugate, Invitrogen, USA) and fibronectin (Rhodamine Fibronectin from bovine plasma; BSF, Cytoskeleton, Inc., USA) were used as model proteins to study their adsorption (or “adherence”) behaviour on the different surfaces by means of fluorescence microscopy using a fluorescence scanner.

Stock solutions of 0.5 mg/ml albumin and 0.5 mg/ml fibrinogen have been made according to the product manuals. For storage these stock solutions were divided into 0.5 ml aliquots and frozen at −20° C. Fibronectin solutions were made directly from the 20 μg vials, without making a stock solution.

All protein-adsorption solutions were made with HEPES 2 buffer prepared with 10 mM 4-(2-hydroxylethyl)-piperazine-1-ethanesulfonic acid (HEPES) and 150 mM NaCl with pH 7.4. Prior to use the HEPES 2 buffer was filtered (Whatman FP 30/0.2 CA-S, size 0.2 μm, maximum pressure 7 bar).

For low protein concentration experiments, the protein solution consisted of filtered HEPES 2 and fluorescently labelled protein of defined concentration (see Table 3 below). For high concentration experiments, unlabelled protein was added additionally to simulate the real protein concentration in human blood (Table 3). To enhance the solubility of unlabelled proteins, HEPES 2 was heated to 37° C. (water bath, INCO 2/108, Memmert GmbH&Co, Germany) prior to the preparation of the solution. The different proteins were tested separately; therefore, the prepared protein solutions always contained only one type of protein. It is assumed that the labelled proteins behave like the unlabelled ones.

To reduce a possible uncertainty in the results due to the instability of the fluorescence marker, the protein solution was freshly prepared right before the adsorption experiments.

The method applied was based on the application of fluorescently labelled proteins and intensity measurements as well as comparison of fluorescence scanning images.

For the albumin and fibrinogen experiments, the samples were generally immersed into 2 ml of protein solution for 10 minutes. The adsorption process was carried out in 24-well plates. Experiments with fibronectin were carried out in 96-well plates and 0.3 ml protein solution but also with an adsorption time of 10 minutes. All adsorption experiments were performed at room temperature.

Proteins not adsorbed onto the surface were removed by submerging the samples in 2 ml of pure HEPES 2 for 10 seconds. Next, they were pivoted in 5 ml of HEPES 2 for 5 seconds followed by a rinsing step with the same 5 ml of HEPES 2. Additionally, the samples were rinsed with ultrapure water for 3 seconds, dried in a stream of nitrogen (at a pressure of about 1 bar) and stored at room temperature in a 24-well plate. To avoid bleaching of the fluorescent label of the adsorbed proteins, the well plates were covered with aluminium foil.

The experimental conditions are given in Table 3 below:

TABLE 3
Experimental conditions of protein adsorption experiments

| | | | | |
|---|---|---|---|---|
| | | Protein | | |
| Protein | Concentration | solution | Time | Samples |

low protein concentration

| | | | | |
|---|---|---|---|---|
| Albumin | 3 μg/mL | 2 mL | 10 min | 6 |
| Fibrinogen | 7 μg/mL | 2 mL | 10 min | 6 |
| Fibronectin | 3 μg/mL | 0.3 mL | 10 min | 6 |

high protein concentration

| | | | | |
|---|---|---|---|---|
| Albumin | 3 μg/mL + 10 mg/mL* | 2 mL | 10 min | 6 |
| Fibrinogen | 7 μg/mL + 1 mg/mL* | 2 mL | 10 min | 6 |
| Fibronectin | 3 μg/mL + 0.2 mg/mL* | 0.3 mL | 10 min | 6 |

*= unlabelled protein

The amount of protein attached to the surface was measured qualitatively using a microarray fluorescence scanner (Axon Genepix 4200A, Molecular Devices, USA). For intensity measurements, the resolution was set to 100 μm/pixel and only one scan per line was performed. For imaging the resolution was set to 5 μm/pixel and three scans per line were performed. To read out the albumin adsorption a laser with a wavelength of 635 nm was used whereas the scanning of fibrinogen and fibronectin adsorbed surfaces was performed using a 532 nm laser. The best focus position was determined separately for every sample.

The photo-multiplier tube (PMT) of the fluorescence scanner was specified to be linear between a gain of 350 to 600. For that reason, all scans were performed in this PMT range. The gain was adapted for each combination of surface, protein and concentration in order to stay in the grey-scale limits of the fluorescence signal of a sample. All gains chosen are listed in Table 4.

TABLE 4
Gains chosen for the RXD SLActive NaOH sample
to measure the protein adsorption

RXD SLActive NaOH

low protein concentration

| | |
|---|---|
| Albumin | 400 |
| Fibrinogen | 350 |
| Fibronectin | 350 |

high protein concentration

| | |
|---|---|
| Albumin | 550 |
| Fibrinogen | 350 |
| Fibronectin | 450 |

To evaluate the homogeneity of the protein adsorption, high-resolution images were compared with each other by visual examination.

The fluorescence intensity data acquired by fluorescence scanning are given in the attached

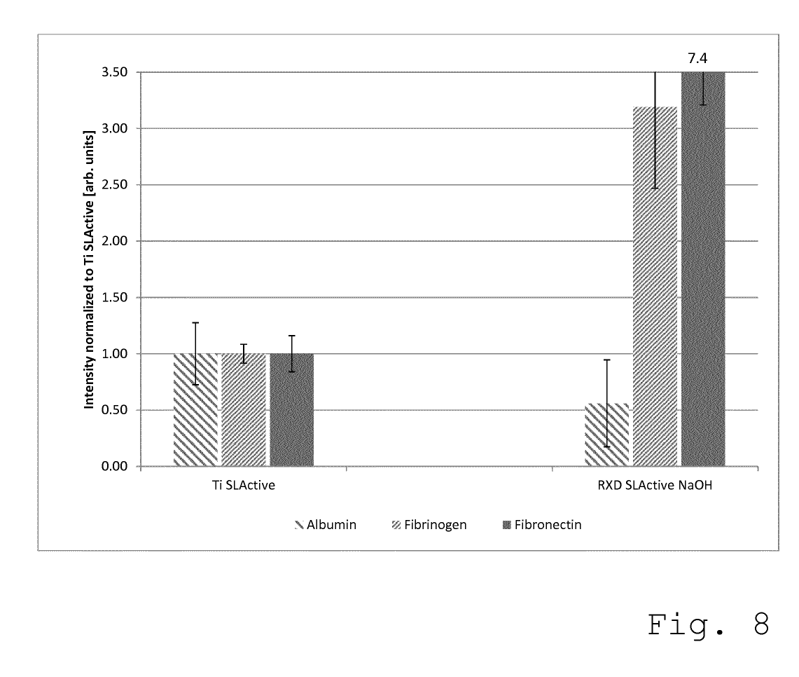

FIG. 8 showing a diagram relating to the fluorescence intensities measured for albumin, fibrinogen and fibronectin on RXD SLActive NaOH surfaces at the low protein concentration defined above; and

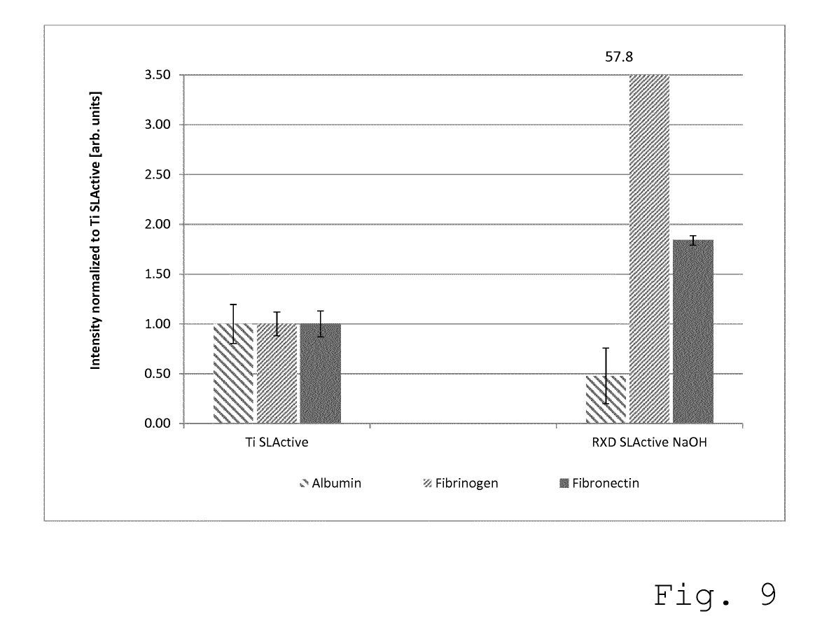

FIG. 9 showing a diagram relating to the fluorescence intensities measured for albumin, fibrinogen and fibronectin on RXD SLActive NaOH surfaces at the high protein concentration defined above.

All values presented in FIGS. 8 and 9 are normalized to the respective intensities measured for a titanium body treated according to the SLActive® protocol (Ti SLActive). The error bars indicate the standard deviation.

According to FIGS. 8 and 9, an extremely high adsorption of the proteins fibrinogen and fibronectin was determined for the RXD SLActive NaOH sample. Whereas at low protein concentration, the improvement in fibrinogen adsorption was more than three times higher than for the Ti SLActive reference, the factor in adsorption improvement was more than 57 at high protein concentration.

Thus, the sample according to the present invention exhibits a highly improved adsorption of the proteins fibrinogen and fibronectin, to both of which an important role in mediating blood coagulation and cell attachment and, thus, tissue interaction with the body is attributed. The adsorption of these proteins is also highly specific, given that the adsorption of non-specific albumin is far lower than for fibrinogen and fibronectin, both at low and high protein concentration.

1.2.5. Assessment of Fibrin Network Formation after Whole Human Blood Incubation

RXD SLA NaOH samples were incubated with whole human blood and analysed for fibrin network formation by SEM and CLSM (confocal laser scanning microscopy) imaging.

Specifically, whole human blood obtained from healthy volunteers was partially heparinized directly with 3 IU/ml sodium heparin (final concentration 0.5 IU heparin/ml blood) and used for the experiments within 1 hour after withdrawal.

Samples were placed into a sample holder and freshly withdrawn blood was added until all samples were covered with a 4 mm thick layer of blood. To prevent further contact with air, the sample holder was closed with a lid and sealed with parafilm before incubation on a tumbling shaker at 10 rpm at room temperature.

The incubation time was determined for each experiment individually. For this, whole blood was spiked with labeled fibrinogen (Alexa488), which allows live monitoring of the blood coagulation on the samples using the fluorescence microscope. As reference, the samples RXD SLA (and RXD SLActive) were used and two time points were chosen (t1: thin, t2: thick fibrin network present on the reference sample).

After incubation, blood was removed and the samples were washed three times by adding pre-warmed PBS into the sample holder, then incubated on a tumbling shaker at 10 rpm for 1 minute for each washing step. Thereafter, the samples were transferred into a new 96-well plate for further treatment.

For SEM imaging, samples were fixed in modified Karnovsky solution for 1 h at room temperature (RT) and then washed twice in PBS. Thereafter, the samples were dehydrated by immersing the samples in solutions of a gradient series of ethanol (50, 70, 80, 90 and 100%), followed by incubation in hexamethyldisilazane (HMDS) for 30 min. Finally, samples were placed into a new 96-well plate and dried overnight at RT. On the next day samples were sputter-coated with gold/palladium (high vacuum coater Leica EM ACE 600, Switzerland). SEM imaging was performed using a Hitachi S-4800 (Hitachi High-Technologies, Canada) at an accelerating voltage of 2 kV and 10 μA current flows.

For CLSM analysis, samples were incubated for 30 min in PBS with 5% goat serum and 1% FCS before staining platelets with Alexa546-labeled phalloidin for 1 h at RT. The platelets and the fibrin network (visible due to spiking of the blood with Alexa488-labeled fibrinogen) were imaged with the CLSM (10×, 40× magnification). Depending on the coverage and thickness of the fibrin network seen by SEM imaging, only one time point (t1 or t2) was imaged. On samples showing complete coverage with fibrin on the surface, the thickness of the fibrin network was measured from z-stack images. To assess the thickness of the fibrin network, 4 z-stack images of 2 samples (2 images per sample with 4 to 6 measurements per image) were analysed to measure the distance from the sample surface to the top surface of the fibrin network using the measure function of the Zeiss ZEN software. CLSM analysis was performed with three independent experiments.

The semi-quantitative analysis of the SEM imaging revealed a trend for higher fibrin network thickness and larger sample coverage of the RXD SLA NaOH sample in comparison to the RXD SLA sample.

This is evidenced by the following figures, of which

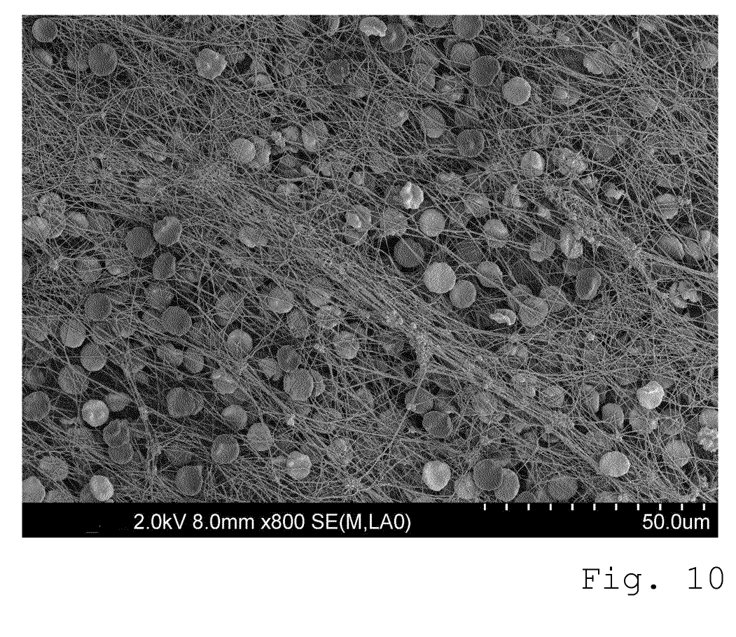

FIG. 10 relates to a SEM image of the surface of sample RXD SLA NaOH after 14 minutes of incubation in a magnification of about 800×, the scale corresponding to 50 micrometer being given in the bottom right corner of the image; and

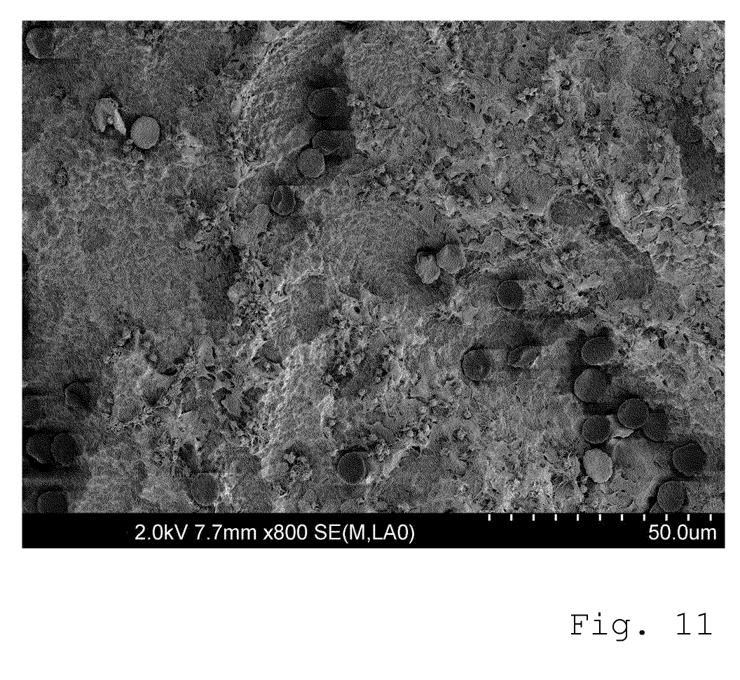

FIG. 11 relates to a SEM image of the surface of sample RXD SLA after 14 minutes of incubation in a magnification of about 800×, the scale corresponding to 50 micrometer being given in the bottom right corner of the image.

Evaluating the presence, distribution and thickness of the fibrin network, the semi-quantitative analysis of the SEM images is summarized in Table 5 showing for four different experiments the value of a qualitative ranking from 0 to 4 taken in each case for two samples and for two different incubation periods given in the table, the ranking starting from patches of blood cells with only few visible fibrin fibers (0) to thick fibrin networks completely covering the sample surface (4).

TABLE 5
Semi-quantitative analysis of the SEM images for four different
experiments performed on RXD SLA NaOH and RXD SLA samples

| | | | |
|---|---|---|---|
| | Incubation | | |
| | period | | |
| Experiment | (min) | RXD SLA NaOH | RXD SLA |

| | | | | | |
|---|---|---|---|---|---|
| 1 | 10 | 2.5 | 2.5 | 1 | 1.5 |
| | 15 | 4 | 4 | 0 | 1 |
| 2 | 12 | 0 | 4 | 0 | 0 |
| | 15 | 4 | 4 | 2 | 2 |
| 3 | 14 | 4 | 3 | 0 | 0 |
| | 17 | 4 | 4 | 0 | 1.5 |
| 4 | 15 | 4 | 4 | 0 | 0 |
| | 18 | 4 | 4 | 0 | 2 |

The thickness of fibrin formed on samples incubated with whole blood (partially heparinized 0.5 IU/ml) for 17 minutes has been assessed as described above, the results of which are given in Table 6.

TABLE 6
Analysis of fibrin layer thickness of RXD SLA NaOH
and RXD SLA samples, the layer thickness was measured,
otherwise, no measurement was done (n.m.)

| Sample | fibrin layer | thickness [μm] |
|---|---|---|
| RXD SLA NaOH | homogenous | 11.02 |
| RXD SLA | none | n.m. |

Thus, in contrast to the RXD SLA samples showing no fibrin network formation, a relatively high fibrin thickness was determined on the samples according to the present invention.

1. Process for the preparation of a topography for improved protein adherence on a body made of titanium or a titanium alloy, the process comprising the subsequent steps of
a) etching the surface with an etching solution comprising a mineral acid, and
b) forming on the etched surface obtained under a) titanate-comprising sub-microscopic structures by treating the etched surface with an aqueous solution comprising an oxidative agent, the sub-microscopic structures extending in at least two dimensions to 1 μm at most.

2. Process according to claim 1, wherein by etching the surface according to step a) a microscopic topographical formation is formed, on which in step b) a layer comprising or essentially consisting of the sub-microscopic structures is formed, the layer forming a sub-microscopic topographical formation.

3. Process according to claim 2, wherein the microscopic topographical formation is defined by at least one of the following surface parameters:
i) Sa being the arithmetic mean deviation of the surface in three dimensions and being in the range from 0.1 μm to 2.0 μm;
ii) St being the maximum peak to valley height of the profile in three dimensions and being in the range from 1.0 μm to 20.0 μm, and/or
iii) Ssk being the skewness of the profile in three dimensions and being in the range from −0.6 to 1.0.

4. Process according to claim 2, wherein by the sub-microscopic topographical formation formed in step b), at least one surface parameter defining the microscopic topographical formation formed in step a) and being selected from the group consisting of Sa, St and Ssk is changed by 50% at most.

5. Process according to claim 1, wherein the sub-microscopic structures extend to a length of more than 50 nm.

6. Process according to claim 1, wherein at least some of the sub-microscopic structures have the shape of a lamella, sheet and/or wire.

7. Process according to claim 6, wherein the thickness of the lamella, sheet and/or wire is in the range from 1 to 50 nm, and the lamella, sheet and/or wire extend in direction perpendicular to the direction of thickness from 50 to 1000 nm.

8. Process according to claim 1, wherein the treatment under step b) is performed under elevated temperatures.

9. Process according to claim 1, wherein the oxidative agent is an inorganic hydroxide.

10. Process according to claim 1, wherein the oxidative agent is a hydroxide of an alkali metal and/or a hydroxide of an alkaline earth metal.

11. Process according to claim 1, wherein the concentration of the oxidative agent is in the range from 0.1 to 5 M.

12. Process according to claim 1, wherein the treatment in step b) is carried out for a duration in the range from 0.25 hours to 12 hours.

13. Process according to claim 1, wherein in step b) the etched surface obtained in step a) is treated with an aqueous solution of sodium hydroxide and/or potassium hydroxide such that sodium titanate-comprising sub-microscopic structures or potassium titanate-comprising sub-microscopic structures are formed, respectively.

14. Process according to claim 1, wherein the body is a dental implant or a dental implant abutment and the topography is provided on at least a portion of the surface of the body that in use is intended to be in contact with hard tissue or soft tissue, respectively.

15. Body obtainable by the process according to claim 1.

16. Body according to claim 15, the surface of which being defined by a microscopic topographical formation and a sub-microscopic topographical formation formed on the microscopic topographical formation, said sub-microscopic topographical formation comprising or essentially consisting of a layer of titanate-comprising sub-microscopic structures extending in at least two dimensions to 1 μm at most.

17. A method comprising using the body according to claim 16 as a dental implant or a dental implant abutment.
